(19) United States
(12) Patent Application Publication
(10) Pub. No.: US 2007/0213795 A1
(43) Pub. Date: Sep. 13, 2007

(54) Implantable medical lead

(76) Inventors: Kerry Bradley, Glendale, CA (US); B. Reno Lauro, Santa Clarita, CA (US)

Correspondence Address: ADVANCED BIONICS CORPORATION, 25129 RYE CANYON ROAD, VALENCIA, CA, 91355, US

(21) Appl. No.: 10/839,518

(22) Filed: May 5, 2004

Related U.S. Application Data
(60) Provisional application No. 60/469,081, filed on May 8, 2003.

(51) Int. Cl. A61N 1/05

(52) U.S. Cl. 607/116; 607/117

(57) ABSTRACT

An improved percutaneous lead is provided. The lead has a circumferential, concave neck located on the distal portion of the lead and a stylet lumen traversing through the lead including through the concave neck. The concave neck has a narrower circumference than the remainder of the lead. The concave neck is designed to bend up to 45 degrees with a pre-curved stylet inserted into the stylet lumen. The presence of a concave neck permits the ingrowth of tissue into and around the concave neck, thereby helping to anchor the lead post-implant. The concavity in the neck presents no sharp edges to disrupt the isodiametric lead profile.

The present application claims the benefit of U.S. Provisional Patent Application Ser. No. 60/469,081, filed 08 May 2003, which application is herein incorporated by reference in its entirety.

### BACKGROUND OF THE INVENTION

The present invention relates to implantable medical leads used to provide electrical stimulation and, more particularly, to steerable, percutaneous leads.

Over the past few decades, significant advances have been made in treating intractable, chronic pain. One method of treating such pain uses an implanted electrode array to stimulate a target area of the spinal cord to alleviate pain. Stimulation of this target area results in paresthesia which can replace the sensation of pain with an alternate, tingling sensation. While the mechanism of pain relief resulting from such electrical stimulation is not well understood, one theory, known as the “Meizack-Wall Gate-Control Theory,” hypothesizes that stimulating specific spinal cord nerves inhibits the conduction through nerve fibers which carry pain signals. Stimulation of these nerve fibers causes inhibition of transmission in the neural pathways leading to the brain which conduct pain signals and, in effect, block the transmission of those pain signals.

Spinal cord stimulation systems generally have two implantable components: an implantable pulse generator (IPG) and leads connected to the outputs of the IPG. The term “lead” will refer to any elongate conductor or conductors, covered with an insulative sheath and having at least one electrode contact connected to the conductor. The lead can have an inner lumen into which a stylet can be inserted. The placement of the stylet inside the lead lumen can help to stiffen the lead so that the lead/stylet combination can be inserted through an introduction cannula needle (which also has a lumen) and through tough, fibrotic tissue. The IPG can be a multi-channel stimulating device encased in a biocompatible container such as titanium. Part of the IPG can also be made from an insulative polymer material such as body-compatible, polyurethane or silicone.

The spinal cord stimulation system can be comprised of one or more leads, each lead having at least one electrode and, more commonly, a multiplicity of electrodes which can be independently selected to provide multipolar stimulation between at least two selected electrodes located on a single lead or, alternatively, between two electrodes, a first electrode located on one lead and a second electrode located on a second lead.

There are several types of leads presently used in spinal cord stimulation. One type is a paddle lead, which has a multiplicity of electrode contacts spaced apart on a flat, paddle-shaped substrate. The paddle lead advantageously allows the electrode contacts to be spaced and shaped to provide wide, bilateral coverage over a stimulation area. A disadvantage presented with a paddle lead is that it requires a laminotomy which is a highly invasive surgical implantation procedure. This surgery can result in significant tissue trauma and subsequent formation of connective scar tissue around the paddle lead and can subject the patient to a substantial risk of infection.

Another type is a percutaneous lead, which can have cylindrical electrode contacts linearly positioned along the distal portion of the head. In contrast to a paddle-type lead, the percutaneous lead generally has a distal end no greater in thickness than the diameter of the remainder of the lead. The percutaneous lead is dimensionally configured for tunneling to a target stimulation site. Using a percutaneous lead avoids some of the disadvantages of using a paddle lead. In particular, no invasive laminotomy is required; the percutaneous lead may be placed through a medium gauge needle reducing surgical trauma. On the other hand, implanting a percutaneous lead also presents its own disadvantages. For instance, a surgeon cannot visually see where the lead is implanted and must rely on imaging technologies such as fluoroscopy to locate the position of the lead during implantation. In addition, a high degree of precision and skill is required to guide the lead and its electrode contacts to the exact stimulation site desired.

A conventional lead implantation procedure commonly places the electrode array parallel to the spinal cord. Precise placement of the electrodes relative to the target nerves is critical for achieving a satisfactory physiological response to spinal cord stimulation and for keeping stimulation thresholds low in order to conserve the IPG's battery power.

One post-implant complication that can occur with spinal cord leads, and particularly percutaneous leads, is that they may migrate, over time, from their initial, implanted position. A lead may become dislodged because of the patient's movement. Such dislodgement often occurs along the rostro-caudal axis, shifting the stimulation field away from the targeted spinal cord segment. This can have the effect of moving paresthesia away from the painful areas of the patient, thus reducing the therapeutic value of the stimulation.

A further challenge with percutaneous leads is that they also be steerable through a cannula and through tough, fibrous tissue. Yet, after the leads are implanted, they must resist migration. In the specific application of spinal cord stimulation, the steerability requirement is often met by making the leads relatively stiff and resistant to buckling. Steerability may also be improved by using a stiff, insertion stylet placed into a lead's lumen. An insertion stylet can be straight or it may have a bent tip. The bent tip may form an angle, typically between 15 to 45 degrees with the stylet axis. Often, however, a stiff stylet cannot bend to a large angle, e.g., bent 45 degrees, because the lead, being relatively stiff, resists such excessive bending at its tip. Because of this natural resistance to bending, a bent stylet inserted into a stiff lead may not provide a clinician with a sufficient tip deflection necessary for adequate steerability. In addition, it is undesirable to use a stylet with large bend angles, such as 45 degrees, because such a highly angled stylet will often jam inside the stiff lead, making withdrawal of the stylet difficult and sometimes causing the lead to be pulled out at the same time. Thus, from a design standpoint, the lead must be sufficiently stiff to resist buckling. Yet, the lead must be sufficiently flexible to allow steering. Once implanted, this steerable lead must resist migration. These represent conflicting design goals which are difficult to achieve simultaneously in a percutaneous lead design.

An implantable lead is disclosed in U.S. Pat. No. 6,185,463 B1, which lead has electrodes placed in recessed areas of the lead. The recessed areas are formed by creating circumferential “notches” where electrode contacts are placed. The notches are shown with sharp angles, which facilitate the manufacturing of the lead but do not facilitate the steerability of the lead because the notches can easily catch at the end of a cannula or on epidural tissue when the lead is being inserted. The lead may also catch on the cannula or the epidural tissue when the lead is being withdrawn.

Accordingly, what is needed is an improved percutaneous lead that exhibits the following characteristics: is easily steered through an introduction cannula and through tough, fibrous tissue, resists buckling during steering, does not require a highly bent stylet, can be easily withdrawn, if explantation is required, and provides adequate anchoring to resist post-implant migration. Such a percutaneous lead can be used for spinal cord stimulation, as well as other stimulation applications, e.g., deep brain stimulation.

### SUMMARY OF THE INVENTION

The present invention addresses the above and other needs by providing an improved percutaneous lead design that accomplishes the above.

In one aspect of the invention, there is provided a percutaneous lead which is substantially isodiametric, except for a concave, circumferential neck located on the distal portion of the lead. The circumferential concavity is smooth and free of sharp edges, particularly at the surface of the lead, which edges can cause the lead to get caught on the discharge end of a cannula during insertion or withdrawal from the cannula. The isodiametric profile of the lead allows the lead to be easily inserted through an introduction cannula and also through tough body tissue. The concavity also advantageously provides a deflection point so that the lead distal tip can bend when a bent stylet is inserted into the lumen of the lead. Additionally, the concave neck of the lead can help to anchor the lead by facilitating the ingrowth of adhesion or scar tissue into and around the concave neck. While anchoring does occur, nevertheless, during explantation, the lead may be pulled from tissue without application of excessive force. The lead design avoids the use of passive protruding structures such as tines or fins which can help fixate the lead long-term but which protruding structures would also render the percutaneous lead unsteerable and make subsequent explantation difficult.

It is thus a feature of the invention to provide a substantially isodiametric, percutaneous lead design that does not utilize obstructive or protruding anchoring structures.

It is another feature of the invention to provide a lead with a concave neck having no sharp edges, which concave neck improves the lead's tip flexibility and improves steerablity and, moreover, facilitates post-implant anchoring.

It is yet another feature of the invention to provide a lead having a tip that is not initially bent, but that is capable of being aggressively angled during use, without requiring that the stylet already be aggressively pre-bent or angled.

### BRIEF DESCRIPTION OF THE DRAWINGS

The above and other aspects, features and advantages of the present invention will be more apparent from the following more particular description thereof, presented in conjunction with the following drawings wherein:

FIG. 1 shows a system view of a typical spinal cord stimulation system;

FIG. 2 shows, a transverse, midsagittal view of a patient's spinal cord and an eight electrode, percutaneous lead placed in parallel to the spinal cord for spinal cord stimulation;

FIG. 3A shows, in accordance with the present invention, a partial view of one embodiment of an improved percutaneous lead, showing the concave neck at the distal end of the lead;

FIG. 3B shows, in accordance with the present invention, a partial view of another embodiment of the percutaneous lead showing button-like electrode contacts and a bullet-shaped tip;

FIG. 4 shows, a cross-sectional view of the lead of FIG. 3A at line 4-4;

FIG. 5 shows, a longitudinal cut-away illustration of the lead shown in FIG. 3A and an inserted stylet.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

FIG. 1 shows a generalized stimulation system that may be used in spinal cord stimulation (SCS), as well as other stimulation application. Such a system typically comprises an implantable pulse generator (“IPG”) 12, an optional lead extension 14, an electrode lead 16 and an electrode array 18. The electrode array includes a plurality of electrode contacts 17 (also referred to herein as “electrodes”). In a percutaneous lead, the electrodes 17 can be arranged in an in-line electrode array 18 near the distal end of the lead 16. Other electrode array configurations can also be used. The IPG 12 generates stimulation current pulses that are applied to selected electrodes 17 within the electrode array 18.

The proximal end of the lead extension 14 can be removably connected to the IPG 12 and a distal end of the lead extension 14 can be removably connected to a proximal end of the electrode lead 16. The electrode array 18 is formed on a distal end of the electrode lead 16. The in-series combination of the lead extension 14 and electrode lead 16 carry the stimulation current from the IPG 12 to electrodes of the electrode array 18. It is noted that the lead extension 14 need not always be used with the neural stimulation system 10. Instead, the lead extension 14 may be used when the physical distance between the IPG 12 and the electrode array 18 requires its use.

The IPG 12 contains electrical circuitry, powered by an internal primary (one-time-use-only) or a rechargeable battery, which electrical circuitry can output current pulses to each stimulation channel. Communication with the IPG can be accomplished using an external programmer (not shown), typically through a radio-frequency (RF) link.

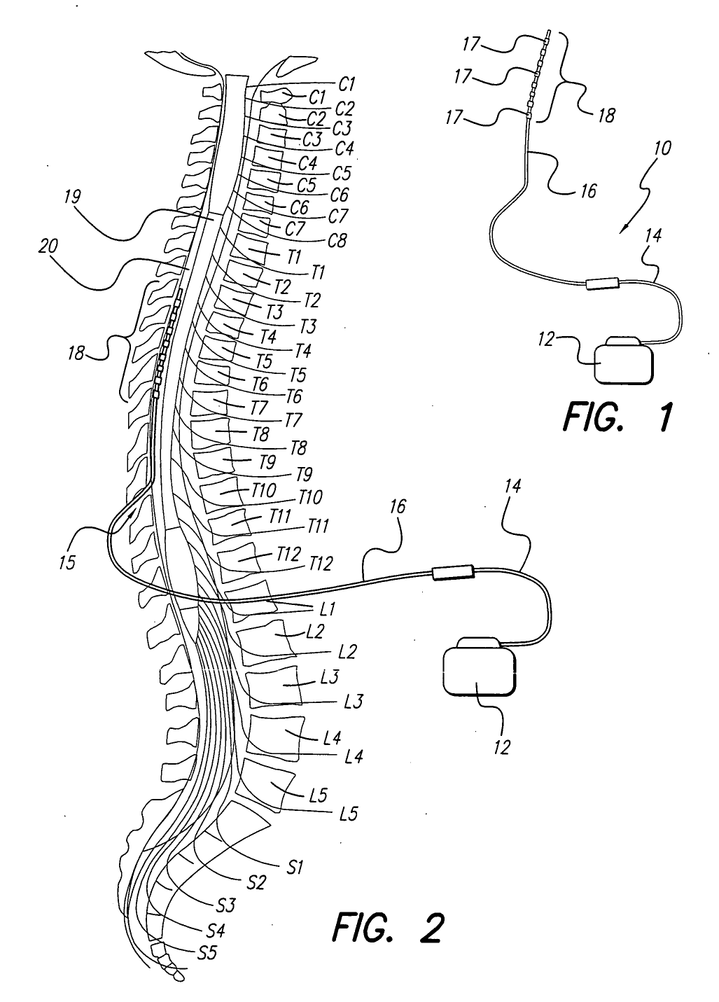

FIG. 2 shows a transverse, midsagittal view of a spinal cord and a generalized, implantable, spinal cord stimulation system. The stimulation system shown is being used as a Spinal Cord Stimulator (SCS) system. In such an application, the lead 16 and, more particularly, the electrode array 18 are implanted in the epidural space 20 of a patient in close proximity to the spinal cord 19. Because of the lack of space near the lead exit point 15 where the electrode lead 16 exits the spinal column, the IPG 12 is generally implanted in the abdomen or above the buttocks. Use of lead extension 14 facilitates locating the IPG 12 away from the lead exit point 15.

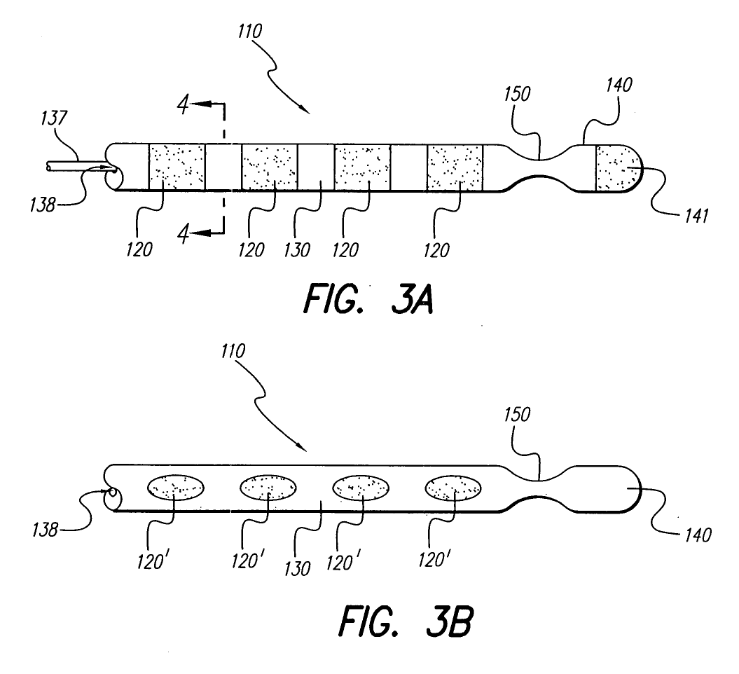

FIG. 3A shows, in accordance with the present invention, a partial view of an improved percutaneous lead 110 that may be used in stimulation applications and, particularly, in an SCS system. Only the distal portion of the lead having the electrodes is shown. The remainder of the lead, including the proximal end, is not shown. A stylet 137 is inserted into lumen 138. The lead 110 has a plurality of electrode contacts 120 placed “in-line” on the lead body 130. The electrode contacts may be made from various body compatible electrode materials such as gold, carbon, platinum and platinum alloys, such as platinum/iridium. The shape of the electrode contacts 120 may be cylindrical or ring-like, as shown in FIG. 3A, and present a smooth profile along the length of the lead 110. The lead 110 has a concave, neck 150 which has a profile that is smooth and is free of sharp edges. A lead tip 140 is at the distal end of the lead 110. With exception of a concave neck 150 and lead tip 140, the lead 110 is preferably substantially isodiametric, meaning that the diameter of the lead along its length is substantially the same. The absence of any sharp edges at the neck 150, combined with the isodiametric profile, prevents the lead 110 from becoming caught on the distal opening wall of a cannula or within tissue during lead insertion or withdrawal, Moreover, the smooth, concave profile of the neck 150 and the isodiametric profile also makes the lead more easily explantable, if that need subsequently arises. In one embodiment of the lead, a plurality of concave necks 150 may be employed, the concave necks 150 variously placed on the distal end of the lead. For example, a concave neck may be placed between two adjacent electrode contacts 120. Alternatively, the concave neck could be, in other lead embodiments, placed somewhere else on the length of the lead 110.

The lead tip 140 may be formed into various shapes such as a multiplanar tip, or more as a bullet, as shown in FIG. 3A and FIG. 3B. In general, the surface configuration of the lead tip 140 is configured to be sharper or narrow than the remainder of the distal portion of the lead in order to easily penetrate fibrous tissue and pass through a cannula. The lead body 130 may be made from silicone, polyurethane or other body implantable polymers, which can act as an insulation over a plurality of conductors (not shown) and wherein each conductor can be connected uniquely to one electrode contact 120. Alternatively, a single conductor may be connected to more than one electrode contact 120. The proximal end of the percutaneous lead 110 may have a connector which is used to electrically and mechanically connect the lead 110 to IPG 12, as shown in FIGS. 1 and 2.

FIG. 3B shows a partial, side view of an alternative embodiment of the lead 110 with a concave neck 150, in accordance with the present invention. The illustration shows only the distal end of the lead 110 having the electrodes 120′ and omits the remainder of the lead body and the proximal end of the lead. FIG. 3B shows button-like or oval electrodes 120′ that are placed on only one side of a percutaneous lead.

The specific embodiments of the lead 110 in FIGS. 3A or 3B shows only four electrodes. However, this is for purposes of illustration only and is not intended to be limiting. In these embodiments, the electrode array may consist of 4, 8 or other number of electrodes arranged in an in-line fashion and can be spaced apart at regular or irregular intervals. The electrode contacts 120, 120′ may be ring-like or cylindrical-like, as shown in FIG. 3A, encircling the entire circumference of the lead, or it may be button-like, occupying only one side of the lead, as seen in FIG. 3B.

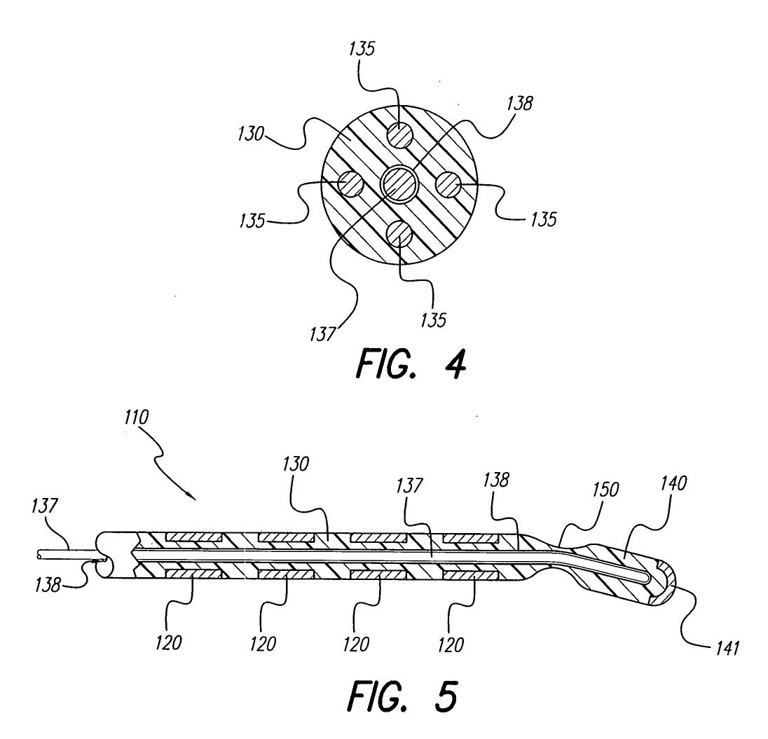

FIG. 4 shows a cross-sectional view of the lead shown in FIG. 3A along line 4-4. FIG. 4 shows an example arrangement of four conductors 135 within the lead body 130. Also shown is the inserted stylet 137 within stylet lumen 138.

FIG. 5 shows a partial, longitudinal, cut-away view of the lead of FIG. 3A with a stylet 137 inserted into the lumen. Only the distal end of the lead 110 is shown in this illustration. The bending occurs at the concave neck 150. This embodiment shows an electrode 141 at the lead tip 140. The electrode 141 and the four electrodes 120 are all connected to conductors that course through the lead body and to the proximal end of the lead (not shown).

The lead 110 can have an stylet lumen 138 which can accommodate a straight or angled insertion stylet 137. The stylet 137 may be angled at its distal end to coincide with the position of the concave neck 150 of the lead 110 so that, when the stylet 137 is fully inserted, the neck 150 becomes bent or angled. Because the neck 150 is narrower than the rest of the lead 110, the neck will bend preferentially before other parts of the lead.

The smoothly curved, concave neck 150 enables the distal end of the lead to easily bend for greater steerability, while employing a less angled stylet. A sufficient angle to the lead tip can be produced using a less angled stylet than normally used because the concave neck 150 is designed to be more flexible. Use of a less angled stylet also allows easier removal of a stylet from the lead without employing excessive pull forces and possibly disengaging the lead from a good stimulating position. The angle of the bend at the concave neck with the lead designs shown in FIGS. 3A and 3B can be from between 0 to about 45 degrees. The lead may be preferably designed to at least provide an bend angle of up to about 25 degrees.

In one embodiment of the lead 110, the lead tip 140 may actually be an electrode or include an electrode 141. Such a tip electrode 141 is depicted in FIG. 3A and FIG. 5.

The concave neck 150 may be formed in various ways. One way of creating this neck is to mold an outer covering of insulation having different thickness. Thus, the insulative covering that forms the outside of the lead body 130 may be thinner at the neck 150 and thicker on the remainder of the lead 110. Additionally, the durometer (hardness) of the insulation may be varied as well, such that the insulative covering at the neck 150 is of lower durometer than the covering over the remainder of the lead. Thus, in addition to employing a concave neck which can bend more easily than the remainder of the lead, the durometer and thickness of the insulative covering may be varied to control the flexibility of the covering and therefore the lead at the concave neck portion.

Adhesion or scar tissue can grow into and around the concave neck 150 after the lead is implanted and help to anchor the lead. The lead design does not use passive protruding structures such as tines or fins which can help to fixate the lead post-implant, but which projections would render the percutaneous lead unsteerable either through a cannula or through tough tissue such as fascia. There is also some concern that such projections could be a focal point for undesirable fibrotic growth and, moreover, mechanically compress nerves causing undesirable physiological consequences, e.g., pain and numbness.

In summary, an improved percutaneous lead is provided which does not use obstructive or protruding structures such as tines or fins. The improved percutaneous lead of the present invention, nevertheless, provides adequate lead fixation by facilitating tissue ingrowth into the concave neck 150. A further advantage of the lead design is that concave neck 150 can be made flexible relative to the remainder of the lead because the neck is smaller in diameter than the remainder of the lead, making the neck a point of bending. In addition, the flexibility of various parts of the lead can be varied by applying different durometer insulation materials over the neck and the remainder of the lead. As thus designed, the percutaneous lead can be easily steered through a cannula and through tissue, can be easily withdrawn from the cannula and from the tissue, and can be subsequently explanted.

While the invention herein disclosed has been described by means of specific embodiments and applications thereof, numerous modifications and variations could be made thereto by those skilled in the art without departing from the scope of the invention set forth in the claims.

1. A medical lead for electrical stimulation comprising:
a lead body;
a plurality of electrodes comprising four or more electrodes;
a distal lead end, extending from the distal end of the lead body; and
a proximal lead end, extending from the proximal end of the lead body,
wherein at least one conductor extends substantially from the distal lead end, through the lead body and to the proximal lead end and wherein an insulative covering covers said at least one conductor; and
wherein the distal end of the lead includes a concave neck that is dimensioned and configured to bend more easily than the remainder of the lead,
wherein the lead also includes a stylet lumen that extends from the proximal lead end, through the lead body, and through the concave neck.

2. The stimulation lead of claim 1, wherein the plurality of electrodes are placed substantially in-line to the lead body.

3. The stimulation lead of claim 2, wherein each electrode is ring-like in shape and encircles the lead body.

4. The stimulation lead of claim 2, wherein each electrode occupies only one side of the lead body and does not encircle the lead body.

5. The stimulation lead of claim 1, wherein the concave neck has surface profile which is gradual and has no sharp edges.

6. The stimulation lead of claim 5, wherein the insulative covering varies in durometer such that the covering has a lower durometer at the concave neck than over the remainder of the lead.

7. The stimulation lead of claim 1, wherein the distal lead end has a lead tip configured to be sharper than the body of the lead to permit easy entry and passage through tissue.

8. The stimulation lead of claim 7, wherein the distal lead tip is shaped like a bullet.

9. The stimulation lead of claim 1, wherein the distal lead end has a lead tip that is an electrode.

10. The stimulation lead of claim 1, wherein the concave neck is one of a plurality of concave necks distributed along the lead length.

11. The stimulation lead of claim 1, wherein the lumen is configured to permit insertion of stylets with preformed bends of varying angles with respect to the stylet axis.

12. The stimulation lead of claim 11, wherein the concave neck is configured to permit a bend angle of from 0 to about 45 degrees, using a stylet with varying bend angles.

13. The stimulation lead of claim 12, wherein the bend angle at the concave neck is between 0 and about 25 degrees.

14. A medical lead for electrical stimulation comprising:
a lead body;
a plurality of electrodes comprising four or more electrodes:
a distal lead end, extending from the distal end of the lead body; and
a proximal lead end, extending from the proximal end of the lead body,
wherein at least one conductor extends substantially from the distal lead end, through the lead body and to the proximal lead end and wherein an insulative covering covers said at least one conductor; and
wherein the distal end of the lead includes a concave neck that is dimensioned and configured to bend more easily than the remainder of the lead,
wherein the lead also includes a stylet lumen that extends from the proximal lead end, through the lead body, and through the concave neck; and
wherein the concave neck is positioned distal to the plurality of electrodes.

15. The stimulation lead of claim 14, wherein the insulative covering varies in durometer such that the covering has a lower durometer at the concave neck than over the remainder of the lead.

16. The stimulation lead of claim 14, wherein the plurality of electrodes are ring-like in shape.

17. A medical lead for electrical stimulation comprising:
a lead body;
a plurality of electrodes:
a distal lead end, extending from the distal end of the lead body; and
a proximal lead end, extending from the proximal end of the lead body,
wherein at least one conductor extends substantially from the distal lead end, through the lead body and to the proximal lead end and wherein an insulative covering covers said at least one conductor; and
wherein the distal end of the lead includes a concave neck that is dimensioned and configured to bend more easily than the remainder of the lead,
wherein the lead also includes a stylet lumen that extends from the proximal lead end, through the lead body, and through the concave neck; and
wherein each electrode occupies only one side of the lead body.

18. The stimulation lead of claim 17, wherein the insulative covering varies in durometer such that the covering has a lower durometer at the concave neck than over the remainder of the lead.
